(19) United States
(12) Patent Application Publication
(10) Pub. No.: US 2020/0100667 A1
(43) Pub. Date: Apr. 2, 2020

(54) Portable device for the measurement of refractive error in the eye

(71) Applicant: Ronak Roy, San Diego, CA (US)

(72) Inventor: Ronak Roy, San Diego, CA (US)

(21) Appl. No.: 16/571,140

(22) Filed: Sep. 15, 2019

Related U.S. Application Data
(60) Provisional application No. 62/739,202, filed on Sep. 29, 2018.

(51) Int. Cl. A61B 3/032; G02F 1/29

(52) U.S. Cl. CPC A61B 3/032; G02F 1/29

(57) ABSTRACT

A portable device for the measurement of refractive error in the eye is disclosed. The device generally includes a liquid lens that the patient looks through, a projective array of static lenses, an electronic display, a system of electronics, and software that controls the potential applied to the liquid lens, controls the display, and runs the algorithm that processes patient feedback in order to determine the correct corrective lens powers. Housed in a singular body that can be held and positioned in front of the patient's eye, the device allows direct feedback and interaction by a patient using a remote control. Altogether, the device is automatically determines a voltage of the liquid lens that provides the patient with the clearest vision, which can be used to calculate the prescription corrective lens power.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

### 1. Field of the Invention

The present invention relates to the field of optics. More particularly, the present invention relates to a portable device for eye examination that determines refractive error.

### 2. Description of the Related Art

Devices used in eye examinations used to measure the refractive error of patient's eyes have been in use for many years. The process of determining the refractive error in a patient's eyes is known in the fields of optometry and ophthalmology as a refraction. One of the most prevalent methods, if not the absolute standard, of refraction is the subjective refraction using the device known as the phoropter.

A traditional phoropter (also known as a refractor) is a device that contains a number of machined static lenses, often made from glass. Phoropters can contain numerous types of lenses, but the two major types are the spherical lenses and the cylindrical lenses.

Spherical lenses are used to diagnose spherical refractive error, which is categorized by a distortion in the shape of the cornea, lens, and/or eye that is symmetric about the central axis that runs perpendicular through the lens. Myopia (also termed near-sightedness) and hyperopia (also termed far-sightedness) are types of spherical refractive error in which light is focused to a region in front of the retina or a region behind the retina, respectively, rather than onto the retina.

Cylindrical lenses are used to diagnose cylindrical refractive error, which is characterized by a misshapen cornea and/or distortion in the lens along an axis that lies in the plane parallel to the retina. Cylindrical refractive error is known as astigmatism, in which light is focused unevenly onto the retina.

To correct for these types of error with prescription eyeglasses or contact lenses, the corrective lenses are shaped with a spherical and/or cylindrical power that counteracts the effects of the error in the eye, such that light arrives at the retina in correct focus. There are many ways to determine the degree of spherical and cylindrical error, with a number of technological ways having been introduced in recent years; however, the subjective refraction using a phoropter remains incredibly common.

In a subjective refraction, the patient looks through the phoropter, a binocular device, with either one or both eyes open, while the medical professional conducting the refraction changes the lens the patient is currently looking through. By having the patient compare two similar, but different, lenses by subjectively determining the clarity of a test chart located approximately six feet away, the examiner can determine the spherical lens power, cylindrical lens power, and axis of cylindrical correction that provides the patient with the clearest vision.

While there have been innovations in recent years that seek to automate and simplify the subjective refractive process, such as motorizing the lenses and providing the eye examiner with a digital control panel, none have addressed a number of key problems with the phoropter, which are enumerated below.

The first problem is the immense cost of the device. Phoropters have a necessity for a large number of precisely machined lenses, produced from an optically clear, refractive material (usually glass) in one-quarter diopter increments. They also require complicated mechanisms that allow the eye examiner to easily and reliably switch between lenses. Both of these requirements lead to incredibly high cost.

The second problem, related to the first, is the sheer size and weight of a standard phoropter. The mechanics, lenses, housing, and other necessary components result in phoropters being immense in volume and in mass. It is common in eye examination offices for the phoropter to be mounted to a massive structure with an arm that holds the device in place in front of the patient's eyes and keeps it stationary once properly positioned. This means that phoropters, once purchased and appropriately configured, are entirely stationary, permanently fixed in the office that they were installed.

The third problem related to a phenomenon in the human eye known as accommodation. When at rest, the human eye's focusing system (comprised of the cornea and lens) is designed to focus light emanating from sources very far away onto the retina. However, for objects that are close to the eye to be put in focus, the focal length of the eye must change; to achieve this, the ciliary muscles in the eye compress the lens in order to increase the curvature, resulting in a decrease in the focal length of the focusing system. While the ability to accommodate reduces with age (resulting in older individuals needing reading glasses, bifocals, or progressive lenses), all humans retain the ability to accommodate to a certain degree throughout their lives, and is especially powerful in children, teenagers, and young adults. As a result, standard eyeglasses are prescribed for far vision, since they eye is capable of automatically increasing its focusing power for nearby objects, but cannot reduce its focusing power past the relaxed state that is supposed to be used for far vision.

As such, eyeglasses must be prescribed with the patient focused on something that is located far away, such that their ciliary muscles will be completely relaxed. While this technically does not happen unless the patient is looking at an object infinitely far away, a distance of six meters (approximately twenty feet) is known as “optical infinity”, or the distance from which light rays emanating from a single point in space are essentially completely parallel, as would be light rays emanating from a point infinitely far away.

Because of this, a typical phoropter requires the patient to focus on a test chart (typically in accordance with the LogMAR standard) that is twenty feet away. This compounds on the lack of portability problem cited earlier, because even if the phoropter could be removed from its permanent mount and transported around, the test chart must be placed a standard distance away, effectively requiring a consistent for testing purposes.

Finally, the process of conducting a refraction is immensely complicated, requiring a trained medical professional to carry out the procedure based on the patient's feedback. This means that even if a reliable means of transporting the expensive and heavy phoropter was developed as well as a reliable means of establishing a consistent exam environment in a location without a permanent optical professional's office, a doctor must still be present to administer the test.

These four problems together present a formidable challenge to the practicality of conducting refractive eye examinations in locations that do not have access to a permanent optometrist's or ophthalmologist's office. Inventions that seek to automate and mechanize the subjective refraction process or conduct objective refractions (refractions that do not rely on the feedback of the patient) fail to address all four of these problems, and continue to restrict access to refractive vision care to wealthy regions of the country and of the world.

In response to the concerns addressed above, the device according to the present invention is a more portable, cheaper, and semi-automated alternative to the classical phoropter and its modern incarnations, all while providing accurate prescriptions for corrective lenses that correct for the refractive error of the human eye.

### BRIEF SUMMARY OF THE INVENTION

In view of the disadvantages inherent in the known types of phoropters now present in the prior art, the present invention provides a new portable device for the measurement of refractive error in the eye that is far less expensive to produce than known phoropters and can be operated and handled completely by the patient and (optionally) a trained practitioner.

The general purpose of the present invention, which will subsequently be described in greater detail, is to provide a new portable device for the measurement of the refractive error in the eye that has many of the advantages of devices used for subjective refractions heretofore and many novel features that result in a new device that is used to conduct subjective refractions which no prior art, alone or in any combination thereof, has anticipated, rendered obvious, suggested, or even implied.

The present invention generally comprises: a liquid lens that the patient looks through, a projective array of static lenses, an electronic display, and a system of electronics and software that controls the potential applied to the liquid lens, controls the display, and runs the algorithm that processes patient feedback in order to determine the correct corrective lens powers. These components of the present invention should be housed in a manufactured or machined housing that can be held and positioned in front of the patient's eye. The would patient interact with the device using a remote control wired to or wireless connected to the electronic and software system.

The preceding description thus outlines, rather broadly, the more important features of the invention in order that the detailed description thereof may be better understood. Additional details and features pertaining to the invention will be described hereinafter and will form the subject matter of the claims. Before explaining features of the invention in detail, it is to be understood that the invention is not limited in its application to the details of construction and to the arrangements of the components specified in the following description or illustrated in the drawings. The invention has other embodiments and may be practiced in other ways not explicitly noted. Also, it is to be understood that the choice of terms and phrases employed herein are for the purpose of the description and should not be regarded as limiting.

An object of the present invention is to provide a device that is used to conduct subjective refractions on patients that uses a programmatically controllable variable focal length liquid lens, rather than a set of static lenses, making the device considerably more lighter, smaller, and less expensive.

Another object of the invention is to provide a device with a set of static lenses positioned such that light emanating from a display mounted to the front of the device is refracted in a way that simulates the screen being much further away than it physically is, thereby replicating the light rays arriving at the corrective liquid lens (before entering the patient's eyes) in a virtually parallel manner.

Another object of the invention is to provide an automated electronics and software system for controlling the lens and the display. The electronics in the invention consist of an integrated circuit that amplifies the input voltage (supplied by a battery) to a range of electric potentials that control the liquid lens and a system comprised of at least one microprocessor that controls the liquid lens driver integrated circuit, controls the graphics (test chart, current voltage, calculated corrective lens power, and other information) depicted on the display, and runs the algorithm that generates pairs of lens voltages, processes the patient's input, and generates the final prescription power for corrective lenses.

The objects of the invention detailed above may be embodied in the form illustrated in the accompanying drawings; however, the drawings provided are solely for the purposes of illustration, and other forms of the invention may be executed while remaining within the scope of the appended claims.

### BRIEF DESCRIPTION OF THE DRAWINGS

FIG. 1 is an isometric view of a housing for an embodiment of the present invention.

FIG. 2 is an isometric view of an optical assembly for an embodiment of the present invention.

### DETAILED DESCRIPTION OF THE INVENTION

### A. Overview

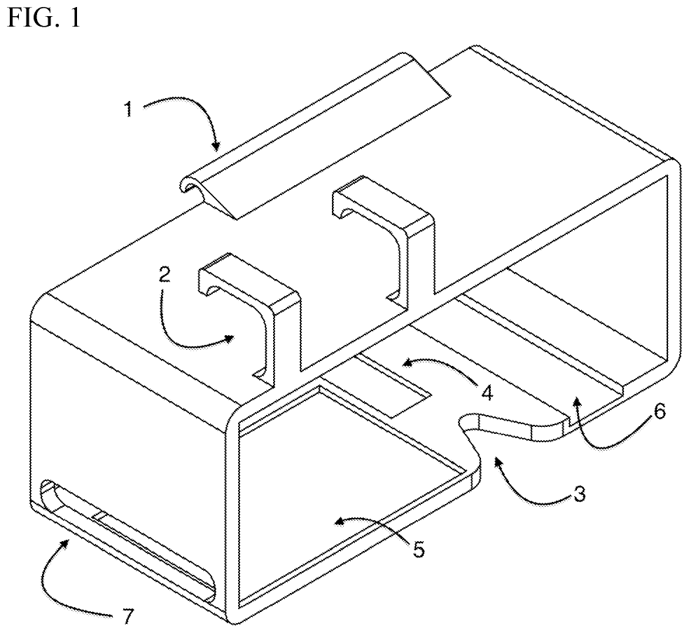

The device contains an optical assembly that the patient looks through to view a display. The display and liquid lens are controlled by the electronics system, which directs the lens driving component of the system to apply certain desired voltages onto the lens, directs the display controlling component of the system to display certain graphics and information, and runs the power determination algorithm. As seen in FIG. 1, other optional modifications to the housing such as a cable passthrough 7 and an inlet for the patient to fit their nose 3.

### B. Optical Assembly

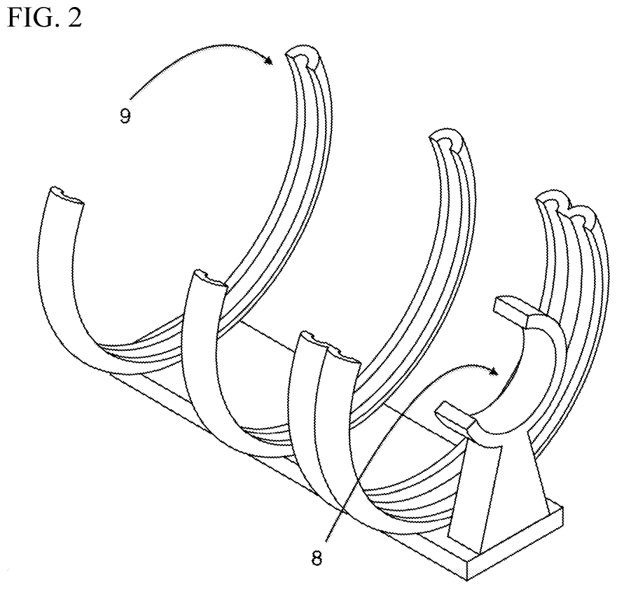

The optical assembly shown in FIG. 2 present in the invention contains a set of at least one static lens, arranged in a specific sequence at calculated distances away from the screen. The lenses are placed into precisely located spots, such as in 9. The purpose of the array of lenses is to project the image of the display to a virtual distance that is much further away from the patient's eyes than in physical reality. Embodiments of the invention may use one of a wide range of distances (with typical virtual distances being fifteen and twenty feet). With the image virtually projected to a distance that simulates the distance between the patient and the screen displaying the test chart in a typical optometrist's or ophthalmologist's office, the light rays emanating from any point of the display will leave the projective array being almost entirely parallel to each other. This serves to relax the accommodation reflex in the eye, such that the ciliary muscles are entirely relaxed so the far vision prescription is accurate.

Embodiments of the invention may opt to use, for the projective array component, the following formula for the effective focal length f of two thin lenses, one with focal lens f₁ and the other with focal length f₂, separated by a distance d:

$$\frac{1}{f} = \frac{1}{{f}_{1}} + \frac{1}{{f}_{2}} - \frac{d}{{f}_{1} {f}_{2}} .$$

In addition, when two thin lenses are placed in sequence, there is a plane known as the real principle plane, which is the virtual location of a single lens with the effective focal length (calculated using the method above) that has the same effect on light rays as the two lenses in sequence. To calculate the distance p between the first lens (the lens through which light passes first) and the real principle plane, the following formula can be used:

$$p = d \frac{f}{{f}_{1}} .$$

Using these equations, any set of lenses can be recursively combined, pair by pair, until it can be effectively treated as a single lens with focal length F, located a distance D from to the front of the array (which can be taken to be the location of the lens closest to the screen) with a certain effective focal length. Once the lens system has been simplified to a single lens using the mathematics described above or some other mathematical treatment of the optical properties of the set of lenses, the following modified version of the thin lens equation may be used to calculate the distance V of the virtual image of the display from the front of the lens assembly, with S being the distance between the display and the front of the projective lens array:

$$\frac{1}{F} = \frac{1}{S + D} - \frac{1}{V + D} .$$

While there are infinitely many combinations of lenses and values of V that yield a virtual image sufficiently far away, a value of fifteen to twenty feet would match the common eye exam office. The methods described above or other methods may be used to calculate the positioning of the lenses relative to the display; however, all will yield the distance between the screen and the real principle plane of the combination to be slightly less than the effective focal length of the system. This yields the result of the light emanating from the display being refracted such that it enters the liquid lens component of the optical assembly virtually parallel.

The other component of the optical assembly is the liquid lens, which would be inserted in the location 8 on FIG. 2. Liquid lenses are devices that contain two transparent, immiscible liquids of different refractive indices, one a polar liquid and the other a nonpolar liquid. When an electric potential is applied across the two liquids, the curvature of the boundary between the two liquids changes. In a liquid lens, these two liquids and electrodes are enclosed in a cylindrical housing with clear apertures on the front and back, such that light passes through and is refracted at the boundary. By applying different voltages, the light will refract to different degrees, effectively resulting in a lens with variable power.

A liquid lens would be located in behind the optical assembly, and during use, the patient looks through the liquid lens as the algorithm that is described hereinafter runs. The optical assembly fits into (or is manufactured directly on) the housing at 6.

### C. Display

Located just beyond the optical assembly (from the patient's perspective) at position 1 in FIG. 1 is the display. The display is mounted either within the housing or outside of the housing, with a clear path such that it can be seen through the optical assembly. The display has a number of responsibilities.

During use, in which an eye test is being conducted on a patient, the display shows a graphic that the patient uses to subjectively determine clarity. The mechanics of an algorithm that may be used in an embodiment of the invention are described hereinafter, but the patient compares at least two lens voltages and provides feedback to the system as to which one makes the graphic appear clearer to them. The graphic may be any image or computer generated graphic; the most common and recommended graphic is a test chart defined by the LogMAR standard, which prescribes rows of optotypes that are logarithmically sized and logarithmically spaced. Each optotype should have strokes that are equal in width, and each letter should be equally legible. It is also recommended that the graphic be frequently switched throughout the test to prevent eye fatigue.

The display should notify the patient that their selection was recorded successfully with a graphic, such as a checkmark.

At the end of the test, the display should read out the final lens voltage and the prescription refractive power for that eye.

### D. Electronics System

In order to control the display and lens, as well as run the algorithm described below, the invention requires an electronic system. There are a number of necessary components of this electronic system.

The Liquid Lens Driver is an integrated circuit (IC) capable of controlling the liquid lens. This IC would input the voltage supplied by a direct-current battery in the device (likely a five volt supply) as well as commands from the Liquid Lens Instructor, and outputs a PWM (pulse-width modulation) signal to the liquid lens such that the root-mean-square voltage is in the range of electrical potentials that allows the lens to modulate its optical power throughout the full range of powers.

The Liquid Lens Controller is a microprocessor-based computing unit that provides the Liquid Lens Driver with commands that correspond to specific desired output voltages. The Liquid Lens Controller, in turn, takes input from the Algorithmic Processing Unit as to exactly what voltages to instruct the Driver to apply to the liquid lens.

The Algorithmic Processing Unit is a microprocessor-based computing unit that runs the Power Determination Algorithm described hereinafter, instructing the Liquid Lens Controller to command the Driver to apply whatever desired voltage the current stage of the algorithm requires. It also takes input directly from the patient, as described in the details of the algorithm.

The Display Controller is a microprocessor-based computing unit that controls the display to display both the reference graphics specified earlier and display data produced by the Algorithmic Processing Unit.

All of these components together serve as the electronics unit of the device. Note that, while they are described as discreet units, they may exist in any combination of the units. For instance, multiple microprocessing units described may be implemented on the same logic board or even within a singular, specialized integrated circuit. Locations for electronics mounting can bee seen in FIG. 1 at positions 4 and 5. They may also share or individually use any set of batteries, which may be located inside the housing or attached to it 2. Portions of the electronics may also be packaged with the display itself, as may be the case in the application of a widescreen mobile phone.

### E. Power Determination Algorithm

As mentioned previously, some component the electronics system must run an algorithm that takes patient feedback in order to, throughout the course of the test, present the patient with lens voltages that provide clearer and clearer vision, until the voltage that provides the subjective clearest vision is found.

Embodiments of the invention may implement one of many variations of the following algorithm. In each cycle, the algorithm should generate a set of lens voltages. Any two voltage in the set should be separated by a certain coarse increment. The patient, by interacting with a button wired to the electronics system or a remote control connected to the system, should be able to switch between each of the voltages in the set; the electronics system should control the liquid lens to apply that voltage. The patient, once inspecting all voltages in the set, should choose the one that provides the clearest vision. Then, the algorithm should use the selected voltage to generate a new set. If the patient selects a voltage that is more extreme than any prior voltage, the new set should contain voltages more extreme than any prior voltage. If the patient selects a voltage that is less extreme than certain previously displayed voltages, this indicates that the algorithm has found an approximate range of voltages that provides the clearest vision. As such, the algorithm should decrease the size of the voltage increment, making the adjustments finer, and generate a new set to sweep the approximate range.

This process should continue until a value that provides the clearest vision is found using a voltage increment that is smaller enough than the patient can no longer discern changes. In practice, this will correspond to an increment in prescription power of approximately one-quarter diopter.

Once this clearest voltage has been determined, it should be used as the input of a calibrated regression which calculates the prescription lens power for that eye of the patient.

### F. Calibration

In order to determine this regression, a number of samples with the embodiment of the invention should be conducted. Patients with a range of refractive error should be tested in both eyes to determine the voltage that provides that eye with that the clearest vision in that specific embodiment of the invention. Patients should also receive prescriptions from a standard phoropter. Then, mathematical regressions can be used on the data, with the independent variable being the voltage and the dependent variable being the prescription power determined by the standard phoropter prescription, which is taken to be the exact value for that patient. The regression will likely be linear, but others fall within the scope of the invention.

Once this regression has been found, the software algorithm can be updated to automatically calculate the prescription power from the final voltage.

I claim:

1. A portable device for the measurement of refractive error in the eye, comprising:
an optical assembly that is made of a liquid lens that the patient looks through followed by a projective array of static lenses,
an electronic display that the patient looks through the optical assembly at,
a system of electronics (accompanied by a battery or batteries), and
software that runs on the electronics system.

2. The portable device for the measurement of refractive error in the eye of claim 1, wherein the components are housed in a manufactured or machined housing.

3. The portable device for the measurement of refractive error in the eye of claim 1, wherein the projective array of static lenses in the optical assembly contains lenses positioned relative to each other and overall is positioned relative to the display to project the image of the display to a virtual distance.

4. The portable device for the measurement of refractive error in the eye of claim 1, wherein the liquid lens in the optical assembly is electronically controllable by the electronics system.

5. The portable device for the measurement of refractive error in the eye of claim 1, wherein the display can show the reference graphic for a patient to compare the clarity of liquid lens voltages.

6. The portable device for the measurement of refractive error in the eye of claim 1, wherein the display can show the voltage and calculated prescription power that provides the patient with the clearest vision.

7. The portable device for the measurement of refractive error in the eye of claim 1, wherein the electronics system controls the potential applied to the liquid lens.

8. The portable device for the measurement of refractive error in the eye of claim 1, wherein the electronics system controls the electronics display.

9. The portable device for the measurement of refractive error in the eye of claim 1, wherein the electronics system runs the algorithm that processes patient feedback in order to determine the liquid lens voltage that provides the clearest vision.

10. The portable device for the measurement of refractive error in the eye of claim 9, wherein the algorithm can be calibrated to calculate a corrective lens prescription power for an eye based on the liquid lens voltage that provides the clearest vision.

11. The portable device for the measurement of refractive error in the eye of claim 10, wherein the algorithm can utilize the calibration data to provide a prescription lens power for the eyes of a patient.

12. The portable device for the measurement of refractive error in the eye of claim 1, wherein a patient can position the device in front of their eye.

13. The portable device for the measurement of refractive error in the eye of claim 1, wherein a patient can provide feedback about the clarity of the lens voltage.

14. The portable device for the measurement of refractive error in the eye of claim 13, wherein the patient's feedback can be entered into the device by either the patient or another person assisting in the operation of the device.
